(19) United States
(12) Patent Application Publication
(10) Pub. No.: US 2017/0151142 A1
(43) Pub. Date: Jun. 1, 2017

(54) HIGH-PERFORMANCE HAIR TREATMENT AGENTS WITH AN ANTI-WASHOUT EFFECT

(71) Applicant: Henkel AG & Co. KGaA, Duesseldorf (DE)

(72) Inventors: Volker Scheunemann, Lueneburg (DE); Erik Schulze zur Wiesche, Hamburg (DE)

(73) Assignee: Henkel AG & Co. KGaA, Duesseldorf (DE)

(21) Appl. No.: 15/359,040

(22) Filed: Nov. 22, 2016

(30) Foreign Application Priority Data
Dec. 1, 2015 (DE) 10 2015 223 831.8

(51) Int. Cl. A61K 8/36; A61K 8/23; A61K 8/898; A61Q 5/00; A61K 8/41; A61K 8/365; A61Q 5/02; A61Q 5/12; A61K 8/34; A61K 8/362

(52) U.S. Cl. CPC A61K 8/36; A61K 8/342; A61K 8/23; A61K 8/898; A61K 8/362; A61K 8/416; A61K 8/365; A61Q 5/02; A61Q 5/12; A61Q 5/004

(57) ABSTRACT

Hair treatment agents, including at least one fatty alcohol, at least one cationic surfactant, at least one bivalent or trivalent metal salt, at least one amino-functional silicone, propionic acid and/or salts of propionic acid, and at least one of organic acid different from propionic acid, reduce or prevent the washing out of color from dyed hair.

### CROSS-REFERENCE TO RELATED APPLICATION

This application claims priority to German Patent Application No. 10 2015 223 831.8, filed Dec. 1, 2015, which is incorporated herein by reference in its entirety.

### TECHNICAL FIELD

This disclosure relates to hair treatment agents, in particular shampoos and so-called conditioners, with an active substance combination for the gentle and effective care of hair.

### BACKGROUND

The importance of care products with the longest-lasting possible effect is increasing not least due to the great stressing of hair, for example, by coloring or permanents, as well as by cleaning the hair with shampoos and by harmful environmental factors. Care products of this type influence the natural structure and properties of hair. Thus, after such care treatments, for example, the wet and dry combability of hair, the hold, and fullness of the hair can be optimized or the hair can be protected from an increase in split ends.

It has long been customary, therefore, to subject hair to a special aftertreatment. In this regard, the hair is treated with special active substances, for example, quaternary ammonium salts or special polymers, usually in the form of a rinse. Depending on the formulation, the combability, hold, and fullness of the hair can be improved by this treatment and the splitting rate reduced.

Multifunctional cosmetic products are also known in the prior art. These include in particular the so-called “2 in 1” shampoos, which not only clean the hair but also condition it. Such products are highly valued by the consumer, because due to their product performance they make at least one process step unnecessary, for example, the conditioning with a classic hair rinse.

Similarly, products for modifying the natural hair color play a prominent role in hair cosmetics. Permanent, semipermanent, or temporary coloring systems are differentiated which are based on chemical and/or natural dyes. The hair colors produced artificially by permanent, semipermanent, or temporary coloring systems have the disadvantage, however, that they can change in an undesirable way, for example, during or after the cleaning of hair.

An “undesirable change” is understood to be the fading or bleeding and the loss of color brilliance of the hair shade achieved by the particular dyeing. Environmental effects and/or the effects of sunlight can intensify these changes still further.

The use of divalent metal salts in hair dyes to improve the durability and thereby the fastness of the color is known from EP 2438900 A1.

There continues to be a need to provide active substances or active substance combinations for hair treatment agents with good care properties, which, moreover, strengthen the adhesion of dyes to hair fibers and thus maintain the fastness of the artificially produced hair color, and in this respect to develop hair treatment agents further.

### BRIEF SUMMARY

Hair treatment agents and methods for treating hair are provided herein. In an embodiment, a hair treatment agent includes:

a) at least one fatty alcohol,
b) at least one cationic surfactant,
c) at least one bivalent or trivalent metal salt.
d) at least one amino-functional silicone,
e) propionic acid and/or salts of propionic acid, and
at least one organic acid different from e).

In another embodiment, a method for treating hair includes applying a hair treatment agent to dry or moist hair. The hair treatment agent includes:
a) at least one fatty alcohol,
b) I at least one cationic surfactant,
c) at least one bivalent or trivalent metal salt,
d) at least one amino-functional silicone,
e) propionic acid and/or salts of propionic acid, and
f) at least one organic acid different from e).
   The hair treatment is agent is left on the hair for a period of 30 to 300 seconds and then rinsed off.

### DETAILED DESCRIPTION

The following detailed description is merely exemplary in nature and is not intended to limit the hair treatment agents and methods for treating hair. Furthermore, there is no intention to be bound by any theory presented in the preceding background or the following detailed description.

It has now been found that a combination of certain ingredients has an especially positive effect on dyed hair treated therewith and on the hair follicle.

A first subject embodiment is hair treatment agents, including

a) at least one fatty alcohol,
b) at least one cationic surfactant,
c) at least one bivalent or trivalent metal salt.
d) at least one amino-functional silicone,
e) propionic acid and/or salts of propionic acid, and
f) at least one organic acid different from e)

Hair treatment agents as contemplated herein are, for example, hair shampoos, hair conditioners, conditioning shampoos, hair sprays, hair rinses, hair treatments, hair packs, hair tonics, permanent wave fixing solutions, hair coloring shampoos, hair dyes, hair setting lotions, hair setting products, hair styling preparations, blow-dry wave lotions, styling mousses, hair gels, hair waxes, or combinations thereof. In view of the fact that men in particular are often reluctant to use multiple different agents and/or multiple application steps, such agents are preferred that a man uses in any event. Preferred agents therefore are shampoos, conditioning agents, or hair tonics.

The hair treatment agents contain at least one fatty alcohol.

Fatty alcohols are aliphatic, long-chain, monohydric primary alcohols with hydrocarbon groups that have 6 to 30, preferably 6 to 22 carbon atoms. The hydrocarbon groups in this case can be saturated or mono- or polyunsaturated. Preferably, as contemplated herein, preferably usable fatty alcohols are selected from 1-hexanediol, 1-heptanol, 1-octanol, 1-decanol, 1-dodecanol (lauryl alcohol), 1-tetradecanol (myristyl alcohol), 1, 1-hexadecanol (cetyl alcohol), 1-heptadecanol (margaryl alcohol), 1-octadecanol (stearyl alcohol), 1-eisosanol (arachidyl alcohol), 1-docosanol (behenyl alcohol), 1-tetracosanol (lignoceryl alcohol), 1-hexacosanol (ceryl alcohol), 1-octacosanol (montanyl alcohol), 1-triacontanol (melissyl alcohol), cis-9-hexadecen-1-ol (palmitoleyl alcohol), cis-9-octadecen-1-ol (oleyl alcohol), trans-9-octadecen-1-ol (elaidyl alcohol), cis-11-octadecen-1-ol, 6,9,12-octadecatrien-1-ol (γ-linolenyl alcohol), and mixtures thereof.

Particularly preferred hair treatment agents are characterized in that, based on their weight, they contain about 0.1 to about 20% by weight, preferably about 0.5 to about 15% by weight, more preferably about 1 to about 10% by weight, and in particular about 2 to about 8% by weight of fatty alcohol(s) of the formula (II)

H₃C—(CH₂)ₖ—CH₂—OH (II),

in which k stands for integers from 4 to 28, preferably from 6 to 24, more preferably from 8 to 22, and in particular for 10, 12, 14, 16, 18, or 20.

Particularly preferred hair treatment agents as contemplated herein include, based on their weight, about 0.1 to about 20% by weight, preferably about 0.5 to about 15% by weight, more preferably about 1 to about 10% by weight, and in particular about 2 to about 8% by weight of alcohol(s) from the group comprising 1-dodecanol (lauryl alcohol), 1-hexadecanol (cetyl alcohol), 1-octadecanol (stearyl alcohol), and 1-docosanol (behenyl alcohol), the amounts referring to the total amount of said fatty alcohols in the composition.

Very particularly preferred hair treatment agents as contemplated herein include, based on their weight, about 0.1 to about 20% by weight, preferably about 0.5 to about 15% by weight, more preferably about 1 to about 10% by weight, and in particular about 2 to about 8% by weight of alcohol(s) from the group of 1-hexadecanol (cetyl alcohol) and 1-octadecanol (stearyl alcohol), the amounts referring to the total amount of said fatty alcohols in the composition.

The hair treatment agents contain at least one cationic surfactant.

The cationic surfactant(s) is/are preferably selected from the group of quaternary ammonium compounds and/or amidoamines, preferred cationic surfactant(s) being selected from
alkyltrimethylammonium chlorides having preferably 10 to 18 carbon atoms in the alkyl group and/or
dialkyldimethylammonium chlorides having preferably 10 to 18 carbon atoms in the alkyl group and/or
trialkylmethylammonium chlorides having preferably 10 to 18 carbon atoms in the alkyl group and/or
cetyltrimethylammonium chloride and/or
stearyltrimethylammonium chloride and/or
behenyltrimethylammonium chloride and/or
distearyldimethylammonium chloride and/or
lauryldimethylammonium chloride and/or
lauryldimethylbenzylammonium chloride and/or
tricetylmethylammonium chloride
Quaternium-27 and/or
Quaternium-83.

Hair treatment agents preferred as contemplated herein include, based on their weight, about 0.05 to about 20% by weight, preferably about 0.1 to about 10% by weight, more preferably about 0.25 to about 8% by weight, and in particular about 0.5 to about 7% by weight of cationic surfactant(s).

Particularly preferred cationic surfactants are selected from compounds of the following formula (I)

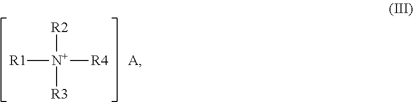

in which at most three R1 to R4 groups independently of one another stand for a saturated or unsaturated, branched or unbranched alkyl group having 1 to 4 C atoms, at least one R1 to R4 group stands for a saturated or unsaturated, branched or unbranched alkyl chain having 8 to 30 C atoms, and A denotes a physiologically acceptable organic or inorganic anion.

In preferred compounds according to formula (I)
two or three R1 to R4 groups stand for a methyl or an ethyl group,
one or two R1 to R4 groups stand for a saturated or unsaturated, branched or unbranched alkyl chain having 14 to 26 C atoms, and
A stands for a halide ion, a sulfate ion of the general formula RSO₃⁻, where R has the meaning of saturated or unsaturated alkyl groups having 1 to 4 carbon atoms, or for an anionic group of an organic acid such as maleic acid, fumaric acid, oxalic acid, tartaric acid, citric acid, lactic acid, or acetic acid.

More preferred are compounds according to formula (III), in which
three R1 to R4 groups stand for a methyl group,
an R1 to R4 group stands for a cetyl, palmityl, stearyl, arachidyl, or a behenyl group, and
A stands for a chloride or a methosulfate ion.

Particularly preferably the at least one compound according to formula (I) is selected from cetyltrimethylammonium chloride, cetyltrimethylammonium methosulfate, behenyltrimethylammonium chloride, and/or behenyltrimethylammonium methosulfate. These compounds can be used in the agent contemplated herein individually or in their combination, wherein the total amount of compounds of the formula (I) in the agent is preferably a maximum of about 10% by weight, and wherein the quantity data refer to the total weight of the agent contemplated herein.

The agent contemplated herein particularly preferably contains behenyltrimethylammonium chloride as the cationic surfactant. Here hair treatment agents contemplated herein are preferred that contain, based on their weight, about 0.05 to about 20% by weight, preferably about 0.1 to about 10% by weight, more preferably about 0.25 to about 8% by weight, and in particular about 0.5 to about 7% by weight of behenyltrimethylammonium chloride.

The hair treatment agents can also contain at least one esterquat as the cationic surfactant.

“Esterquats” as contemplated herein are preferably understood to be compounds of the following formula (IV)

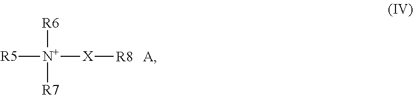

in which
the R5, R6, and R7 groups each independently of one another can be identical or different and have the following meaning:
a saturated or unsaturated, branched or unbranched alkyl group having 1 to 4 carbon atoms which can contain at least one hydroxyl group, or
a saturated or unsaturated, branched or unbranched or a cyclic saturated or unsaturated alkyl group having 6 to 30 carbon atoms which can contain at least one hydroxyl group, or
an aryl or alkylaryl group, for example, phenyl or benzyl, or
(—X—R8), with the proviso that at most 2 of the R5, R6, or R7 groups can stand for (—X—R8),
   wherein
   X has the following meaning:
—(CH2)n- with n=1 to 20, preferably n=1 to 10, and particularly preferably n=1 to 5, or
—(CH₂—CHR9-O)ₙ— with n=1 to 200, preferably 1 to 100, more preferably 1 to 50, and particularly preferably 1 to 20, and with R9 having the meaning of hydrogen, methyl, or ethyl, or
a hydroxyalkylene group having one to four carbon atoms, which may be branched or unbranched and which contains at least one and at most 3 hydroxyl groups, and wherein R8 has the following meaning:
R10-O—CO—, where R10 is a saturated or unsaturated, branched or unbranched or a cyclic saturated or unsaturated alkyl group having 6 to 30 carbon atoms, which can contain at least one hydroxyl group, and which optionally can be oxethylated furthermore with 1 to 100 ethylene oxide units and/or 1 to 100 propylene oxide units, or
R11-O—CO—, where R11 is a saturated or unsaturated, branched or unbranched or a cyclic saturated or unsaturated alkyl group having 6 to 30 carbon atoms, which can contain at least one hydroxyl group, and which optionally can be oxethylated furthermore with 1 to 100 ethylene oxide units and/or 1 to 100 propylene oxide units, or
   in which A stands for a physiologically acceptable organic or inorganic anion, preferably one of the R5, R6, or R7 groups stands for the group (—X—R8), R8 stands for a non-ethoxylated fatty acid group, such as for a palmitic, stearic, arachidic, or behenic acid group, in particular a stearic acid group, and A stands for a halide ion, a sulfate ion of the general formula RSO₃⁻, where R has the meaning of saturated or unsaturated alkyl groups having 1 to 4 carbon atoms, or for an anionic group of an organic acid such as maleic acid, fumaric acid, oxalic acid, tartaric acid, citric acid, lactic acid, or acetic acid, in particular for a chloride ion or for a methosulfate ion.

The esterquats suitable for the agents contemplated herein are preferably selected from at least one of the products marketed under the trade names Rewoquat®, Stepantex®, Dehyquart®, Armocare®, and Akypoquat®. Specific examples as contemplated herein of particularly suitable esterquats are the products: Armocare® VGH-70, Dehyquart® F-75, Dehyquart® C-4046, Dehyquart® L80, Dehyquart® F-30, Dehyquart® AU-35, Rewoquat® WE18, Rewoquat® WE38 DPG, Stepantex® VS 90, and Akypoquat® 131.

Agents contemplated herein are particularly preferred that contain as the esterquat at least one of the compounds known under the INCI names Distearoylethyl Hydroxyethylmonium Methosulfate and Distearoylethyl Hydroxyethylmonium Chloride.

Distearoylethyl Hydroxyethylmonium Methosulfate is particularly preferred, which can be contained in the agents contemplated herein in a preferred amount of about 0.1 to about 10% by weight, more preferably of about 0.5 to about 8% by weight, particularly preferably of about 0.75 to about 6% by weight, and in particular of about 1 to about 5% by weight, the quantitative data referring to the total weight of the agent contemplated herein.

The esterquat(s) can be added to the agents contemplated herein both individually and also as a mixture with other active care substances.

Because of the better manageability and processability, it can be of advantage if the esterquat(s), in particular Distearoylethyl Hydroxyethylmonium Methosulfate, is/are added to the agents contemplated herein as an active substance mixture. An especially suitable example of such an active substance mixture is obtainable, for example, under the trade name Dehyquart® F 75 from the company BASF (Distearoylethyl Hydroxyethylmonium Methosulfate and Cetearyl Alcohol).

The hair treatment agents contain at least one bivalent or trivalent metal salt.

To achieve an optimal action, it is of advantage if the metal salts are present in dissolved form in the agents contemplated herein. In a preferred embodiment, the hair cleaning agents and hair care agents contemplated herein therefore contain water-soluble bivalent or trivalent metal salts. “Water-soluble” is understood here to mean that at least 1 g of the particular salt can be dissolved completely in 1 L of water at 20° C.

Suitable divalent or trivalent metal salts can be selected from divalent or trivalent organic and/or inorganic salts. If propionates are used, these are included in component e) and are not taken into account in the calculation of component c) (bivalent or trivalent metal salt).

Especially suitable cations within these salts can be selected preferably from alkaline earth metal cations and from copper, zinc, iron(II), iron(III), and/or aluminum cations.

Alkaline earth metal cations are very particularly preferred and calcium and magnesium cations are particularly preferred. Especially suitable organic anions within these salts can be selected preferably from acetate, lactate, succinate, citrate, tartrate, malate, maleate, oxalate, and/or glycolate ions. Acetate, lactate, and/or citrate salts with the aforementioned cations are very especially preferred.

Particularly preferred organic salts are calcium lactate, calcium citrate, calcium acetate, magnesium lactate, magnesium citrate, and/or magnesium acetate.

Particularly suitable inorganic anions within these salts can be selected from halide, sulfate, phosphate, and/or carbonate ions. Sulfate and/or halide ions such as chloride and bromide ions are very particularly preferred.

Particularly preferred inorganic salts are calcium chloride, calcium sulfate, magnesium chloride, and/or magnesium sulfate.

The weight proportion of the at least one bivalent or trivalent metal salt in terms of the total weight of the hair treatment agents contemplated herein is preferably about 0.01 to about 10% by weight, primarily about 0.1 to about 7.5% by weight, more preferably about 0.2 to about 5% by weight, and in particular about 0.3 to about 3% by weight.

Hair treatment agents preferred as contemplated herein include, based on their weight, about 0.01 to about 10% by weight, preferably about 0.1 to about 7.5% by weight, more preferably about 0.2 to about 5% by weight, and in particular about 0.3 to about 3% by weight of at least one bivalent or trivalent metal salt from the group of organic or inorganic copper, zinc, iron(II), calcium, magnesium, iron(III), and/or aluminum salts.

The water-soluble salts are particularly preferred in this embodiment. Calcium lactate, calcium citrate, calcium acetate, magnesium lactate, magnesium citrate, magnesium acetate, calcium halides, calcium hydroxide, magnesium halides, and/or magnesium hydroxide are very particularly preferred in this embodiment

The hair treatment agents contain at least one amino-functional silicone. Such silicones can be described, e.g., by the formula

M(RₐQ_{b}SiO_{(4-a-b)/2)x}(R_{c}SiO_{(4-c)/2)y}M,

wherein R in the above formula is a hydrocarbon or a hydrocarbon group having 1 to about 6 carbon atoms, Q is a polar group of the general formula —R¹HZ, wherein R¹ is a divalent linking group that is bound to hydrogen and the Z group and is made up of carbon and hydrogen atoms, carbon, hydrogen, and oxygen atoms or carbon, hydrogen, and nitrogen atoms, and Z is an organic, amino-functional group that contains at least one amino-functional group; “a” assumes values in the range of about 0 to about 2, “b” assumes values in the range of about 1 to about 3, “a”+“b” is less than or equal to 3, and “c” is a number in the range of about 1 to about 3, and x is a number in the range of 1 to about 2000, preferably of about 3 to about 50, and most preferably of about 3 to about 25, and y is a number in the range of about 20 to about 10,000, preferably of about 125 to about 10,000, and most preferably of about 150 to about 1000, and M is a suitable silicone end group, as is known in the state of the art, preferably trimethylsiloxy. Non-limiting examples of the groups represented by R include alkyl groups, such as methyl, ethyl, propyl, isopropyl, isopropyl, butyl, isobutyl, amyl, isoamyl, hexyl, isohexyl, and the like; alkenyl groups such as vinyl, halovinyl, alkylvinyl, allyl, haloallyl, and alkylallyl; cycloalkyl groups such as cyclobutyl, cyclopentyl, cyclohexyl, and the like; phenyl groups, benzyl groups, halohydrocarbon groups such as 3-chloropropyl, 4-bromobutyl, 3,3,3-trifluoropropyl, chlorocyclohexyl, bromophenyl, chlorophenyl, and the like, and sulfur-containing groups such as mercaptoethyl, mercaptopropyl, mercaptohexyl, mercaptophenyl, and the like; R is preferably an alkyl group, containing 1 to about 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—, and —(CH₂)₃C(O)SCH₂CH₂—.

Z is an organic, amino-functional group containing at least one functional amino group. A possible formula for Z is NH(CH₂)_{z}NH₂, where z is 1 or more. Another possible formula for Z is —NH(CH₂)_{z}(CH₂)_{zz}NH, where both z and zz are independently 1 or more, this structure comprising diamino ring structures such as piperazinyl. Z is most preferably a —NHCH₂CH₂NH₂ group. Another possible formula for Z is —N(CH₂)_{z}(CH₂)_{zz}NX₂ or —NX₂, where each X of X₂ is selected independently from the group consisting of hydrogen and alkyl groups having 1 to 12 carbon atoms, and zz is 0.

Q is most preferably a polar, amino-functional group of the formula —CH₂CH₂CH₂NHCH₂CH₂NH₂. In the formulas, “a” assumes values in the range of about 0 to about 2, “b” assumes values in the range of about 2 to about 3, “a”+“b” is less than or equal to 3, and “c” is a number in the range of about 1 to about 3. The molar ratio of the RₐQ_{b} SiO_{(4-a-b)/2} units to the R_{c}SiO_{(4-c)/2} units is in the range of about 1:2 to 1:65, preferably from about 1:5 to about 1:65, and most preferably from about 1:15 to about 1:20. If one or more silicones of the above formula are used, then the various variable substituents in the above formula can be different in the various silicone components, present in the silicone mixture.

Preferred agents contemplated herein are characterized in that they contain an amino-functional silicone of the formula (Si-II)

R′ₐG₃₋ₐ—Si(OSiG₂)ₙ—(OSiG_{b}R′_{2-b})ₘ—O—SiG₃₋ₐ—R′ₐ (Si-II),

in which:
-G is —H, a phenyl group, OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a stands for a number between 0 and 3, in particular 0;
b stands for a number between 0 and 1, in particular 1,
m and n are numbers whose sum (m+n) amounts to between 1 and 2000, preferably between 50 and 150, n preferably assuming values from 0 to 1999 and in particular from 49 to 149 and m preferably assuming values from 1 to 2000, in particular from 1 to 10,
R′ is a monovalent group selected from
   -Q-N(R″)—CH₂—CH₂—N(R″)₂
   -Q-N(R″)₂
   -Q-N⁺(R″)₃A⁻
   -Q-N⁺H(R″)₂A⁻
   -Q-N⁺H₂(R″)A⁻
   -Q-N(R″)—CH₂—CH₂—N⁺R″H₂A⁻,
      wherein each Q stands for a chemical bond, —CH₂—, —CH₂—CH₂—, —CH₂CH₂CH₂—, —C(CH₃)₂, —CH₂CH₂CH₂CH₂—, —CH₂C(CH₃)₂—, —CH(CH₃)CH₂CH₂—,
      R″ stands for identical or different groups from the group —H, -phenyl, -benzyl, CH₂—CH(CH₃)Ph, C₁₋₂₀ alkyl groups, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion, which is preferably selected from chloride, bromide, iodide, or methosulfate.

Particularly preferred agents contemplated herein are characterized in that they contain at least one amino-functional silicone of the formula (Si-IIa)

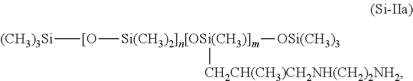

in which m and n are numbers whose sum (m+n) amounts to between 1 and 2000, preferably between 50 and 150, n preferably assuming values from 0 to 1999 and in particular from 49 to 149 and m preferably assuming values from 1 to 2000, in particular from 1 to 10.

These silicones are designated as Trimethylsilylamodimethicone in accordance with the INCI Declaration.

Particularly preferred as well are agents contemplated herein that contain an amino-functional silicone of the formula (Si-IIb)

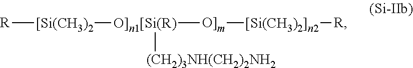

in which R stands for —OH, —O—CH₃, or a —CH₃ group and m, n1 and n2 are numbers, whose sum (m+n1+n2) amounts to between 1 and 2000, preferably between 50 and 150, the sum (n1+n2) preferably assuming values from 0 to 1999 and in particular from 49 to 149 and m preferably assuming values from 1 to 2000, in particular from 1 to 10.

These silicones are designated as Amodimethicone in accordance with the INCI Declaration.

Irrespective of which amino-functional silicones are used, agents contemplated herein are preferred that contain an amino-functional silicone whose amine value is above 0.25 meq/g, preferably above 0.3 meq/g, and in particular above 0.4 meq/g. The amine value here stands for the milliequivalents of amine per gram of the amino-functional silicone. It can be determined by titration and may also be given in the unit mg of KOH/g.

Hair treatment agents preferred as contemplated herein are characterized in that, based on their weight, they contain about 0.01 to about 20% by weight, preferably about 0.1 to about 10% by weight, more preferably about 0.5 to about 7.5% by weight, and in particular about 1 to about 5% by weight of amino-functional silicone(s).

The agents contemplated herein contain with particular preference amino-functional silicone(s) with terminal hydroxy group(s). Some special amino-functional silicone(s) with terminal hydroxy group(s) have emerged as being especially suitable in the agents contemplated herein. These are described hereafter.

Agents preferred as contemplated herein are characterized in that, based on their weight, they contain about 0.01 to about 20% by weight, preferably about 0.1 to about 10% by weight, particularly preferably about 0.5 to about 7.5% by weight, and in particular about 1 to about 5% by weight of at least one silicone of the following formula (Si-III)

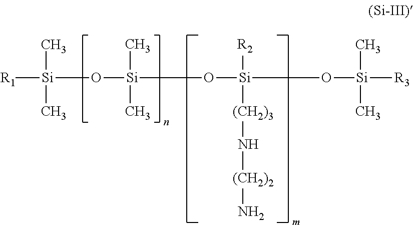

in which
m and n denote numbers that are selected such that the sum (n+m) is in the range of 1 to 1000,
n is a number in the range of 0 to 999 and m is a number in the range of 1 to 1000,
R1, R2, and R3, which are identical or different, denote a hydroxy group or a C1-4 alkoxy group,
at least one of the R1 to R3 groups denoting a hydroxy group.

Further agents preferred as contemplated herein are characterized in that, based on their weight, they contain about 0.01 to about 20% by weight, preferably about 0.1 to about 10% by weight, particularly preferably about 0.5 to about 7.5% by weight, and in particular about 1 to about 5% by weight of at least one silicone of the following formula (Si-IV)

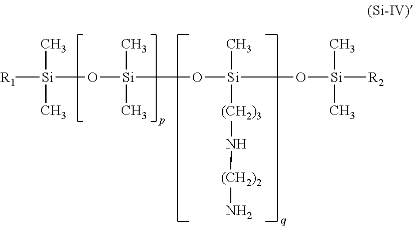

in which
p and q denote numbers that are selected such that the sum (p+q) is in the range of 1 to 1000,
p is a number in the range of 0 to 999 and q is a number in the range of 1 to 1000,
R1 and R2, which are different, denote a hydroxy group or a C1-4 alkoxy group, at least one of the R1 to R2 groups denoting a hydroxy group.

The silicones of the formulas (Si-III) and (Si-IV) differ by the grouping on the Si atom that carries the nitrogen-containing group: In formula (Si-III), R2 denotes a hydroxy group or a C1-4 alkoxy group, whereas the group in formula (Si-IV) is a methyl group. The individual Si groupings, which are labeled with the subscripts m and n or p and q, need not be present as blocks, but rather the individual units can also be distributed randomly; i.e., in the formulas (Si-III) and (Si-IV) each R1-Si(CH₃)₂ group is not necessarily bound to a —[O—Si(CH₃)₂] grouping.

Pretreatment agents that contain at least one silicone of the formula (Si-V) have emerged as especially effective in the method contemplated herein in regard to the desired effects:

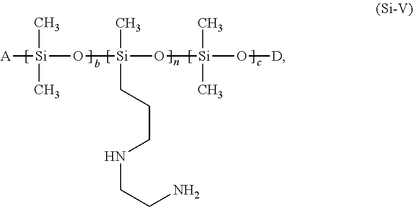

in which
A stands for a group —OH, —O—Si(CH₃)₃, —O—Si(CH₃)₂OH, —O—Si(CH₃)₂OCH₃,
D stands for a group —H, —Si(CH₃)₃, —Si(CH₃)₂OH, —Si(CH₃)₂OCH₃,
b, n and c stand for integers between 0 and 1000,
   provided that
   n>0 and b+c>0
   at least one of the conditions A=—OH or D=—H is met.

Hair treatment agents contemplated herein that contain, based on their weight, about 0.01 to about 20% by weight, preferably about 0.1 to about 10% by weight, particularly preferably about 0.5 to about 7.5% by weight, and in particular about 1 to about 5% by weight of at least one silicone of the formula (Si-V):

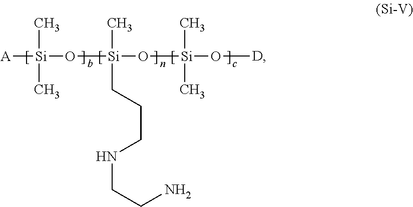

in which
A stands for a group —OH, —O—Si(CH₃)₃, —O—Si(CH₃)₂OH, —O—Si(CH₃)₂OCH₃,
D stands for a group —H, —Si(CH₃)₃, —Si(CH₃)₂OH, —Si(CH₃)₂OCH₃,
b, n and c stand for integers between 0 and 1000,
provided that
n>0 and b+c>0
at least one of the conditions A=—OH or D=—H is met,
   are accordingly preferred as contemplated herein.

In the aforementioned formula (Si-V), the individual siloxane units with the subscripts b, c, and n are randomly distributed; i.e., they need not necessarily be block copolymers.

Further particularly suitable silicones are 4-morpholinomethyl-substituted. Hair treatment agents contemplated herein are particularly preferred that contain, based on their weight, about 0.01 to about 20% by weight, preferably about 0.1 to about 10% by weight, particularly preferably about 0.5 to about 7.5% by weight, and in particular about 1 to about 5% by weight of at least one 4-morpholinomethyl-substituted silicone of the formula (Si-VI),

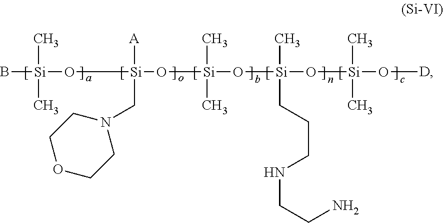

in which
A stands for a structural unit (i) bound via an —O—

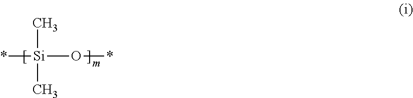

or for an oligomeric or polymeric group bound via an —O— and containing structural units of the formula (i) or for —OH,

* stands for a bond to the structural unit (i) or for an end group B (Si-bound) or D (O-bound),
B stands for a group —OH, —O—Si(CH₃)₃, —O—Si(CH₃)₂OH, —O—Si(CH₃)₂OCH₃,
D stands for a group —H, —Si(CH₃)₃, —Si(CH₃)₂OH, —Si(CH₃)₂OCH₃,
a, b and c stand for integers between 0 and 1000, with the proviso that a+b+c>0
m, n, and o stand for integers between 1 and 1000,
with the proviso that at least one of the conditions B=—OH or D=—H is met.

Structural formula (Si-VI) is intended to clarify that the siloxane groups n and o need not necessarily be bound directly to an end grouping B or D. Instead, in preferred formulas (Si-VI) a>0 or b>0 and in particularly preferred formulas (Si-VI) a>0 and b>0; i.e., the terminal grouping B or D is preferably bound to a dimethylsiloxy grouping. In formula (Si-VI) as well, the siloxane units a, b, c, n, and o are preferably distributed randomly.

The silicones represented by formula (Si-VI) and used as contemplated herein can be trimethylsilyl-terminated (D or B=—Si(CH₃)₃), but they may also be dimethylsilylhydroxy-terminated at both ends or dimethylsilylhydroxy- and dimethylsilylmethoxy-terminated at one end. In the context as contemplated herein, silicones used with particular preference are selected from silicones in which

B=—O—Si(CH₃)₂OH and D=—Si(CH₃)₃
B=—O—Si(CH₃)₂OH and D=—Si(CH₃)₂OH
B=—O—Si(CH₃)₂OH and D=—Si(CH₃)₂OCH₃
B=—O—Si(CH₃)₃ and D=—Si(CH₃)₂OH
B=—O—Si(CH₃)₂OCH₃ and D=—Si(CH₃)₂OH.
These silicones lead to enormous improvements in the hair properties of hair treated with the agents contemplated herein, in particular to greatly improved protection during oxidative treatment.

In formula (Si-VI) as well, the A group can stand
for a structural unit (i) bound via an —O— or
an oligomeric or polymeric group bound via an —O— and containing structural units of the formula (i)
or for —OH.

Thus, formula (Si-VI) is more precisely one of the formulas (Si-VIa), (Si-VIb), or (Si-VIc):

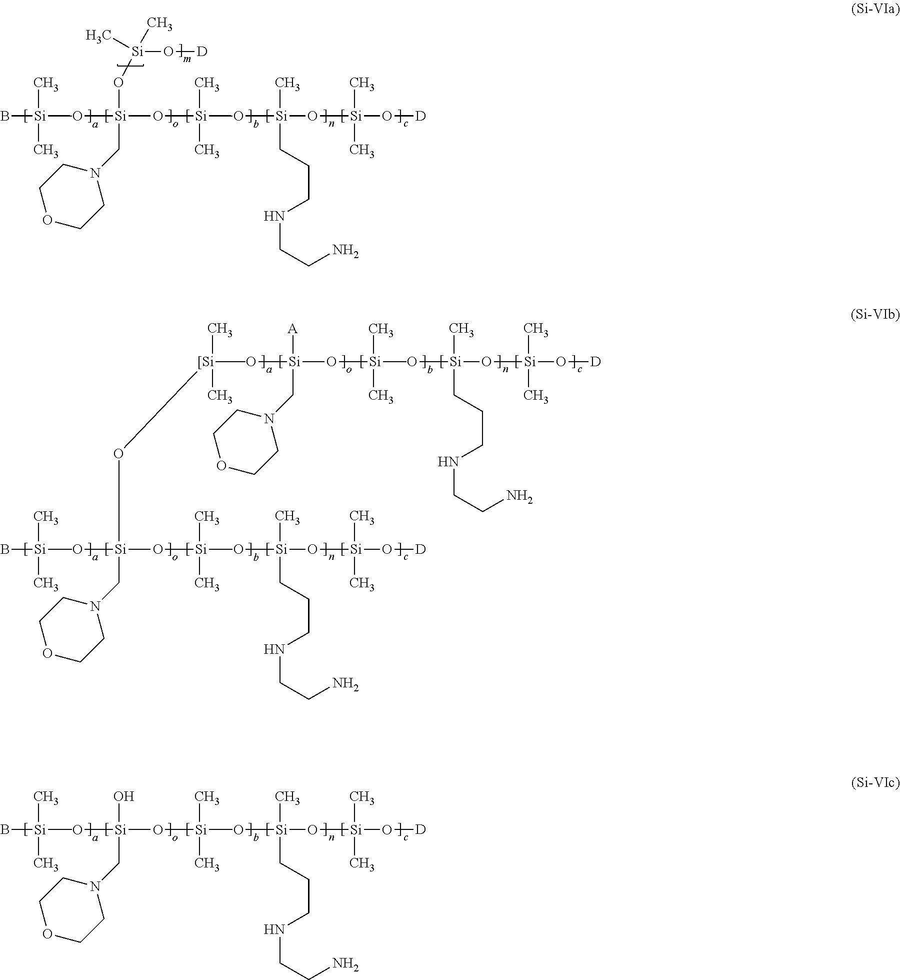

Irrespective of the type of employed amino-functional silicone(s) with terminal hydroxy group(s), the agents contemplated herein contain the silicone(s) preferably in the form of an emulsion, particularly preferably in the form of a microemulsion.

It has been found that the action of the silicones used in the agents contemplated herein can be increased still further if specific nonionic components are also used in the agents. In addition, these nonionic components have positive effects on the storage stability of the agents. Nonionic components that are particularly suitable here are ethoxylates of decanol, undecanol, dodecanol, tridecanol, etc. Ethoxylated tridecanols which are incorporated more preferentially into the agents contemplated herein have proven particularly suitable. Particularly preferred agents as contemplated herein are characterized in that, based on their weight, they contain about 0.00001 to about 5% by weight, preferably about 0.0001 to about 3.5% by weight, particularly preferably about 0.001 to about 2% by weight, more preferably about 0.01 to about 1% by weight, and in particular about 0.1 to about 0.5% by weight of branched, ethoxylated tridecanol (INCI name: Trideceth-5) or α-isotridecyl-w-hydroxypolyglycolether (INCI name: Trideceth-10), or mixtures thereof.

The hair treatment agents contain propionic acid and/or salt(s) of propionic acid. Propionic acid is used with preference, wherein use within specific narrow amount ranges is especially effective.

Preferred hair treatment agents contemplated herein contain, based on their weight, about 0.001 to about 4% by weight, preferably about 0.01 to about 3% by weight, more preferably about 0.025 to about 2.5% by weight, and in particular about 0.05 to about 2% by weight of propionic acid.

Salts of propionic acid can also be used in addition to propionic acid or in its place. The alkali metal salts, inter alia sodium propionate, potassium propionate and ammonium propionate, magnesium propionate, calcium propionate, zinc propionate, iron propionate, and manganese propionate, have emerged as especially suitable here. Preferred hair treatment agents contemplated herein contain salt(s) from the group comprising sodium propionate, potassium propionate and ammonium propionate, magnesium propionate, calcium propionate, zinc propionate, iron propionate, and manganese propionate in a total amount of about 0.001 to about 0.4% by weight, preferably about 0.01 to about 0.3% by weight, more preferably about 0.025 to about 0.25% by weight, and in particular about 0.05 to about 0.2% by weight, based in each case on the weight of the agent.

Particularly preferred hair treatment agents contemplated herein contain, based on their weight, about 0.001 to about 4% by weight, preferably about 0.01 to about 3% by weight, more preferably about 0.025 to about 2.5% by weight, and in particular about 0.05 to about 2% by weight of salt(s) from the group comprising sodium propionate, potassium propionate, and calcium propionate.

The hair treatment agents contain at least one organic acid different from propionic acid. Typical representatives of organic acids are aliphatic mono- and dicarboxylic acids such as, for example, acetic acid, oxalic acid, and 1,3-propanedioic acid and aromatic carboxylic acids such as, for example, benzoic acid. Other organic acids are, for example, hydroxycarboxylic acids such as glycolic acid, citric acid, tartaric acid, malic acid, and lactic acid. Unsaturated mono- or dicarboxylic acids such as, for example, fumaric acid, or α-ketocarboxylic acids such as, for example, pyruvic acid (2-oxopropionic acid) are included as contemplated herein.

Hair treatment agents preferred as contemplated herein include, based on their weight, about 0.001 to about 10% by weight, preferably about 0.01 to about 5% by weight, more preferably about 0.05 to about 3% by weight, and in particular about 0.1 to about 2% by weight of organic acid(s) different from propionic acid.

Particularly preferred hair treatment agents contain, based on their weight, 0.001 to 10% by weight, preferably about 0.01 to about 5% by weight, more preferably about 0.05 to about 3% by weight, and in particular about 0.1 to about 2% by weight of organic acid(s) from the group comprising formic acid, acetic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluic acid, hydratropic acid, atropic acid, cinnamic acid, isonicotinic acid, nicotinic acid, dicarbamic acid, 4,4′-dicyano-6,6′-dinicotinic acid, 8-carbamoyloctanoic acid, 1,2,4-pentanetricarboxylic acid, 2-pyrrolecarboxylic acid, 1,2,4,6,7-naphthalenepentaacetic acid, malonaldehydic acid, 4-hydroxyphthalamidic acid, 1-pyrazolecarboxylic acid, gallic acid, or propanetricarboxylic acid, glycolic acid, lactic acid, malic acid, tartaric acid, or citric acid.

Some representatives are particularly preferred from among the aforementioned acids, because they impart in addition a pleasant shine to hair treated with agents contemplated herein. Most preferred hair treatment agents contain, based on their weight, about 0.001 to about 10% by weight, preferably about 0.01 to about 5% by weight, more preferably about 0.05 to about 3% by weight, and in particular about 0.1 to about 2% by weight of organic acid(s) from the group comprising formic acid, oxalic acid, maleic acid, lactic acid, tartaric acid, or citric acid.

The hair treatment agents contain the above-described active substances preferably in a cosmetic acceptable carrier. In the context contemplated herein, this is understood to be preferably an aqueous or aqueous-alcoholic carrier.

The cosmetic carrier contains preferably at least about 50% by weight, more preferably at least about 60% by weight, particularly preferably at least about 70% by weight, and especially preferably at least about 75% by weight of water.

Furthermore, the cosmetic carrier can contain about 0.01 to about 40% by weight, preferably about 0.05 to about 30% by weight, and in particular about 0.1 to about 20% by weight of at least one alcohol.

Suitable alcohols are, for example, ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol, phenoxyethanol, or mixtures of said alcohols.

The water-soluble alcohols are particularly preferred. Ethanol, 1,2-propylene glycol, glycerol, benzyl alcohol, and mixtures of said alcohols are particularly preferred.

It is of advantage for a very good (scalp) skin compatibility of the hair treatment agents contemplated herein if these have a slightly acidic pH.

It has been found that the agents contemplated herein have an especially good skin compatibility and mildness in a pH range of about 4.2 to about 5.8.

In a first preferred embodiment, the hair treatment agents contemplated herein therefore preferably have a pH in the range of about 4.2 to about 5.8, more preferably from about 4.25 to about 5.6, particularly preferably from about 4.3 to about 5.5, exceedingly preferably from about 4.35 to about 5.4, and especially preferably from about 4.4 to about 5.3.

The hair treatment agents contemplated herein may contain vegetable oils, vegetable butters, and/or vegetable waxes. These vegetable oil components give the hair an improved combability and stylability and increase the hair shine.

Suitable vegetable oil components include natural (vegetable) oils and/or butter, which typically contain triglycerides and mixtures of triglycerides.

Preferred natural oils are coconut oil, (sweet) almond oil, walnut oil, peach kernel oil, apricot kernel oil, argan oil, avocado oil, tea tree oil, soybean oil, sesame oil, sunflower oil, tsubaki oil, evening primrose oil, rice bran oil, palm kernel oil, mango kernel oil, marula oil, cuckoo flower oil, thistle oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter, and/or shea butter.

Carnauba wax, beeswax, and/or candelilla wax can be used with preference as suitable natural or vegetable waxes.

Particularly preferred vegetable oil components are (sweet) almond oil, peach kernel oil, apricot kernel oil, amaranth seed oil, argan oil, olive oil, jojoba oil, cocoa butter, and/or shea butter. Apricot kernel oil, argan oil, olive oil, and/or jojoba oil are particularly preferred.

In a preferred embodiment, the hair treatment agents contemplated herein preferably contain coconut oil, (sweet) almond oil, walnut oil, peach kernel oil, apricot kernel oil, argan oil, avocado oil, tea tree oil, soybean oil, sesame oil, sunflower oil, tsubaki oil, evening primrose oil, rice bran oil, palm kernel oil, mango kernel oil, marula oil, cuckoo flower oil, thistle oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter, and/or shea butter.

Within this embodiment, it is particularly preferable if the hair treatment agents contemplated herein contain (sweet) almond oil, peach kernel oil, apricot kernel oil, amaranth seed oil, olive oil, jojoba oil, cocoa butter, and/or shea butter.

The weight proportion of the at least one vegetable oil, vegetable butter, and/or vegetable wax in terms of the total weight of the hair treatment agents contemplated herein is preferably about 0.02 to about 2.50% by weight, more preferably of about 0.03 to about 2.00% by weight, particularly preferably about 0.04 to about 1.50% by weight, and in particular about 0.05 to about 1.00% by weight.

Apart from the aforementioned essential and facultative components, the hair treatment agents contemplated herein in a further preferred embodiment for a further increase in the care properties of the agents can contain at least one further active hair-conditioning substance, which can be selected from the group of
protein hydrolysates,
vitamins,
plant extracts, and/or
glycerol.

Suitable protein hydrolysates are understood to be product mixtures that can be obtained by acid-, base-, or enzyme-catalyzed degradation of proteins.

Protein hydrolysates of plant, animal, and/or marine origin can be used.

Animal protein hydrolysates are, for example, elastin, collagen, keratin, silk, and milk protein hydrolysates, which can also be present in the form of salts. Such products are marketed, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), and Kerasol® (Croda).

Preferred are protein hydrolysates of plant origin, e.g., soybean, almond, rice, pea, potato, and wheat protein hydrolysates. Such products are available, for example, under the tradenames Gluadin® (Cognis), DiaMin® (Diamalt), Lexein® (Inolex), and Crotein® (Croda). Cationized protein hydrolysates can also be used, wherein the underlying protein hydrolysate may originate from animals, for example, from collagen, milk, or keratin, from plants, for example, from wheat, corn, rice, potatoes, soybeans, or almonds, from marine life forms, for example, from fish collagen or algae, or from biotechnologically obtained protein hydrolysates. The protein hydrolysates underlying the cationic derivatives may be obtained from the corresponding proteins by chemical, in particular, alkaline or acidic hydrolysis, by enzymatic hydrolysis, and/or by a combination of both types of hydrolysis. The hydrolysis of proteins usually gives rise to a protein hydrolysate with a molecular weight distribution of about 100 Da up to several thousand daltons. Preferred cationic protein hydrolysates are those whose underlying protein portion has a molecular weight of 100 up to 25,000 Da, preferably 250 to 5000 Da. Furthermore, cationic protein hydrolysates are understood to be quaternized amino acids and mixtures thereof. Quaternization of the protein hydrolysates or of the amino acids is often performed by means of quaternary ammonium salts such as, for example, N,N-dimethyl-N-(n-alkyl)-N-(2-hydroxy-3-chloro-n-propyl)ammonium halides. Furthermore, the cationic protein hydrolysates can also be derivatized still further. Mentioned as typical examples of the cationic protein hydrolysates and derivatives are the products that are commercially available and are known under the INCI names: Cocodimonium Hydroxypropyl Hydrolyzed Collagen, Cocodimopnium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxyproypltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, and Quaternium-79 Hydrolyzed Wheat Protein.

The weight proportion of the protein hydrolysate(s) in terms of the total weight of the hair treatment agents is preferably about 0.01 to about 5% by weight, more preferably about 0.025 to about 3% by weight, and in particular about 0.05 to about 2% by weight.

Irrespective of the source (plant, animal, marine, etc.), protein hydrolysates depending on the degree of hydrolysis contain individual amino acids, oligopeptides, and optionally polypeptides.

The hair treatment agents contemplated herein contain with particular preference at least one oligopeptide that has at least one amino acid sequence Glu-Glu-Glu, wherein the amino group may be present in a free or protonated form and the carboxy groups in a free or deprotonated form.

Preferred hair treatment agents contemplated herein are characterized in that, based on their weight, they contain 0.0001 to 10% by weight of at least one oligopeptide that has at least one amino acid sequence Glu-Glu-Glu, wherein the amino group may be present in a free or protonated form and the carboxy groups in a free or deprotonated form.

In this formula, as in all formulas below, the amino group hydrogen atom in parentheses like the acid function hydroxy group in parentheses means that the groups in question may be present as such (in which case it is an oligopeptide with the particular number of amino acids as shown (in the present formula 3)) or, however, that the amino acid sequence is present in an oligopeptide that comprises still further amino acids (depending on where the further amino acid(s) is/are bound, the components in parentheses in the above formula are replaced by the further amino acid group(s)).

These preferred hair treatment agents contemplated herein contain, based on their weight, about 0.0001 to about 10% by weight of at least one oligopeptide that has at least one amino acid sequence Glu-Glu-Glu, i.e., at least three consecutive glutamic acids.

Oligopeptides in the context of the present application are amino acid condensation products which are linked acid amide-like by peptide bonds and comprise at least 3 and a maximum of 25 amino acids.

In preferred hair treatment agents contemplated herein, the oligopeptide comprises 5 to 15 amino acids, preferably 6 to 13 amino acids, particularly preferably 7 to 12 amino acids, and in particular 8, 9 or 10 amino acids.

Depending on whether additional amino acids are bound to the Glu-Glu-Glu sequence and depending on the type of said amino acids, the molar mass of the oligopeptide present in the agents contemplated herein may vary. Preferred hair treatment agents used as contemplated herein are characterized in that the oligopeptide has a molar mass of about 650 to about 3000 Da, preferably of about 750 to about 2500 Da, particularly preferably of about 850 to about 2000 Da, and in particular of about 1000 to about 1600 Da.

In summary, preferred hair treatment agents are characterized in that the oligopeptide comprises 5 to 15 amino acids, preferably 6 to 13 amino acids, particularly preferably 7 to 12 amino acids, and in particular 8, 9, or 10 amino acids and has a molar mass of about 650 to about 3000 Da, preferably of about 750 to about 2500 Da, particularly preferably of about 850 to about 2000 Da, and in particular of about 1000 to about 1600 Da.

As can be seen from the preferred number of amino acids in the oligopeptides and the preferred molar mass range, oligopeptides consisting not only of the three glutamic acids but also having additional amino acids bound to this sequence are used with preference. These additional amino acids are preferably selected from specific amino acids, whereas specific other representatives are less preferred as contemplated herein.

It is thus preferred, if the oligopeptides used in the agents contemplated herein do not contain any methionine.

It is preferred further, if the oligopeptides used in the agents contemplated herein do not contain any cysteine and/or cystine.

It is preferred further, if the oligopeptides used in the agents contemplated herein do not contain any aspartic acid and/or asparagine.

It is preferred further, if the oligopeptides used in the agents contemplated herein do not contain any serine and/or threonine.

In contrast, it is preferred, if the oligopeptides used in the agents contemplated herein contain tyrosine.

It is preferred further, if the oligopeptides used in the agents contemplated herein contain leucine.

It is preferred further, if the oligopeptides used in the agents contemplated herein contain isoleucine.

It is preferred further, if the oligopeptides used in the agents contemplated herein contain arginine.

It is preferred further, if the oligopeptides used in the agents contemplated herein contain valine.

Particularly preferred oligopeptides or amino acid sequences contained in the preferred oligopeptides are described hereafter:

A particularly preferred oligopeptide additionally contains tyrosine, which is bound preferably via its acid function to the Glu-Glu-Glu sequence. Hair treatment agents preferred as contemplated herein are therefore characterized in that the oligopeptide contained therein has at least one amino acid sequence Tyr-Glu-Glu-Glu, wherein the amino group may be present in a free or protonated form and the carboxy groups in a free or deprotonated form.

A further particularly preferred oligopeptide additionally contains isoleucine, which is bound preferably via its amino function to the Glu-Glu-Glu sequence. Hair treatment agents preferred as contemplated herein are therefore characterized in that the oligopeptide contained therein has at least one amino acid sequence Tyr-Glu-Glu-Glu, wherein the amino group may be present in a free or protonated form and the carboxy groups in a free or deprotonated form.

Oligopeptides that have both of the aforesaid amino acids (tyrosine and isoleucine) are preferred contemplated herein. Particularly preferred in this case are hair treatment agents contemplated herein in which the oligopeptide contained in the hair treatment agent has at least one amino acid sequence Tyr-Glu-Glu-Glu-Ile, wherein the amino group may be present in a free or protonated form and the carboxy groups in a free or deprotonated form.

Further preferred oligopeptides additionally contain arginine, which is preferably present bound to isoleucine.

Particularly preferred in this case are hair treatment agents contemplated herein in which the oligopeptide contained in the hair treatment agent has at least one amino acid sequence Tyr-Glu-Glu-Glu-Ile-Arg, wherein the amino groups may be present in a free or protonated form and the carboxy groups in a free or deprotonated form.

Still further preferred oligopeptides additionally contain valine, which is preferably present bound to arginine. Hair treatment agents preferred further as contemplated herein are therefore characterized in that the oligopeptide contained in the hair treatment agent has at least one amino acid sequence Tyr-Glu-Glu-Glu-Ile-Arg-Val, wherein the amino groups may be present in a free or protonated form and the carboxy groups in a free or deprotonated form.

Still further preferred oligopeptides additionally contain leucine, which is preferably present bound to valine. Hair treatment agents preferred further as contemplated herein are therefore characterized in that the oligopeptide contained in the hair treatment agent has at least one amino acid sequence Tyr-Glu-Glu-Glu-Ile-Arg-Val-Leu, wherein the amino groups may be present in a free or protonated form and the carboxy groups in a free or deprotonated form.

Particularly preferred oligopeptides additionally contain leucine, which is preferably present bound to tyrosine. Hair treatment agents preferred further as contemplated herein are therefore characterized in that the oligopeptide contained in the hair treatment agent has at least one amino acid sequence Leu-Tyr-Glu-Glu-Glu-Ile-Arg-Val-Leu, wherein the amino groups may be present in a free or protonated form and the carboxy groups in a free or deprotonated form.

Very particularly preferably, the agents contemplated herein contain at least two oligopeptides that meet the aforesaid criteria, but differ from one another. Thus, for example, the use of hair treatment agents is preferred that contain at least two different oligopeptides A and B, both of which contain the amino acid sequence Glu-Glu-Glu.

Such different oligopeptides A and B correspond to one another in that they have three consecutive Glu amino acids in their amino acid sequence, but have differences in the amino acids bound before or after these. Different peptides with a partial agreement, which may be much greater than in the aforementioned three amino acids, are preferred.

Thus, further preferred hair treatment agents are characterized in that the hair treatment agent contain at least two different oligopeptides A and B, both of which contain the amino acid sequence Glu-Glu-Glu-Ile.

Likewise preferred are hair treatment agents that contain at least two different oligopeptides A and B, both of which contain the amino acid sequence Tyr-Glu-Glu-Glu.

Even further preferred hair treatment agents are characterized in that they contain at least two different oligopeptides A and B, both of which contain the amino acid sequence Glu-Glu-Glu-Ile-Arg.

Likewise further preferred hair treatment agents are characterized in that the hair treatment agents contain at least two different oligopeptides A and B, both of which contain the amino acid sequence Tyr-Glu-Glu-Glu-Ile.

Preferred hair treatment agents contemplated herein are therefore characterized in that the oligopeptide has at least one Tyr-Glu-Glu-Glu-Ile amino acid sequence, wherein the amino group may be present in a free or protonated form and the carboxy groups in a free or deprotonated form.

Very particularly preferred hair treatment agents are characterized in that the hair treatment agent contains at least two different oligopeptides A and B, both of which contain the amino acid sequence Glu-Glu-Glu-Ile-Arg.

Likewise very particularly preferred hair treatment agents are characterized in that the hair treatment agent contains at least two different oligopeptides A and B, both of which contain the amino acid sequence Tyr-Glu-Glu-Glu-Ile-Arg.

Preferably, there is an even greater structural agreement in the oligopeptides. Thus, hair treatment agents that contain at least two different oligopeptides A and B, both of which contain the amino acid sequence Glu-Glu-Glu-Ile-Arg-Val, are further preferred embodiments as contemplated herein.

Also preferred embodiments are hair treatment agents that contain at least two different oligopeptides A and B, both of which contain the amino acid sequence Tyr-Glu-Glu-Glu-Ile-Arg-Val.

Even further preferred hair treatment agents contemplated herein are characterized in that they contain at least two different oligopeptides A and B, both of which contain the Glu-Glu-Glu-Ile-Arg-Val-Leu amino acid sequence.

Likewise still further preferred hair treatment agents contemplated herein are characterized in that they contain at least two different oligopeptides A and B, both of which contain the amino acid sequence Tyr-Glu-Glu-Glu-Ile-Arg-Val-Leu.

Preferred hair treatment agents contemplated herein are therefore characterized in that the oligopeptide has at least one amino acid sequence Tyr-Glu-Glu-Glu-Ile-Arg-Val-Leu, wherein the amino groups may be present in a free or protonated form and the carboxy groups in a free or deprotonated form.

Particularly preferred hair treatment agents contemplated herein are characterized in that they contain at least two different oligopetides A and B, wherein oligopeptide A has the amino acid sequence Leu-Tyr-Glu-Glu-Glu-Ile-Arg-Val-Leu, wherein the amino groups may be present in a free or protonated form and the carboxy groups in a free or deprotonated form and the oligopeptide B has the amino acid sequence Tyr-Glu-Glu-Glu-Ile-Arg-Val-Leu, wherein the amino groups may be present in a free or protonated form and the carboxy groups in a free or deprotonated form.

The very particularly preferred hair treatment agents of this last embodiment contain, based on the weight of the agent, about 0.00001 to about 1% by weight of oligopeptide A and about 0.00001 to about 1% by weight of oligopeptide B.

The further preferred hair treatment agents of this last embodiment contain, based on the weight of the agent, about 0.00005 to about 0.1% by weight of oligopeptide A and about 0.00005 to about 0.1% by weight of oligopeptide B.

The still further preferred hair treatment agents of this last embodiment contain, based on the weight of the agent, about 0.0001 to about 0.01% by weight of oligopeptide A and about 0.0001 to about 0.001% by weight of oligopeptide B.

The oligopeptides used in the context as contemplated herein, which meet the aforesaid conditions, can be obtained advantageously from keratinic materials. It is preferred contemplated herein that these oligopeptides are used in large amounts, based on the total keratinic peptide content of the agents.

It is very particularly preferred that the greatest possible amount of all keratinic peptides contained in the agent contemplated herein meets the aforesaid conditions.

Preferred hair treatment agents contemplated herein are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides, contained in the agent, have the amino acid sequence Glu-Glu-Glu.

Additionally preferred hair treatment agents contemplated herein are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides, contained in the agent, have the amino acid sequence Glu-Glu-Glu-Ile.

Even more preferred hair treatment agents contemplated herein are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides, contained in the agent, have the amino acid sequence Tyr-Glu-Glu-Glu.

Particularly preferred hair treatment agents contemplated herein are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides, contained in the agent, have the amino acid sequence Tyr-Glu-Glu-Glu-Ile.

Very particularly preferred hair treatment agents contemplated herein are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides, contained in the agent, have the amino acid sequence Tyr-Glu-Glu-Glu-Ile-Arg.

Even more particularly preferred hair treatment agents contemplated herein are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides, contained in the agent, have the amino acid sequence Tyr-Glu-Glu-Glu-Ile-Arg-Val.

Particularly preferred hair treatment agents contemplated herein are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides, contained in the agent, have the amino acid sequence Tyr-Glu-Glu-Glu-Ile-Arg-Val-Leu.

The aforesaid conditions relate to the total content of peptides, originating from keratinic materials, in the agents contemplated herein. In addition to the oligopeptides of keratinic origin, naturally further peptides and/or protein hydrolysates can be used, for example, from other native sources. For example, the additional use of wheat protein hydrolysates is preferred.

Suitable vitamins are understood preferably as the following vitamins, provitamins, and vitamin precursors, and derivatives thereof:
Vitamin A: the group of substances designated as vitamin A include retinol (Vitamin A₁) and 3,4-didehydroretinol (vitamin A₂). β-Carotene is the retinol provitamin. Suitable vitamin A components are, for example, vitamin A acid and esters thereof, vitamin A aldehyde, and vitamin A alcohol and esters thereof such as the palmitate and the acetate.
Vitamin B: The vitamin B group or the vitamin B complex includes, inter alia,
vitamin B₁ (thiamine)
vitamin B₂ (riboflavin)
vitamin B₃. The compounds nicotinic acid and nicotinamide (niacinamide) are often included under this term.
vitamin B₅ (pantothenic acid and panthenol). Panthenol is preferably used within the scope of this group. Usable panthenol derivatives are particularly the esters and ethers of panthenol, pantolactone, and cationically derivatized panthenols. Individual representatives are, for example, panthenol triacetate, panthenol monoethyl ether, and the monoacetate thereof, as well as cationic panthenol derivatives.
vitamin B₆ (pyridoxine as well as pyridoxamine and pyridoxal).
Vitamin C (ascorbic acid): The use in the form of the palmitic acid ester, glucosides, or phosphates can be preferred. The use in combination with tocopherols can likewise be preferred.
Vitamin E (tocopherols, especially α-tocopherol).
Vitamin F: The term “vitamin F” is conventionally understood to mean essential fatty acids, in particular linoleic acid, linolenic acid, and arachidonic acid.
Vitamin H: Vitamin H is the name for the compound (3aS,4S,6aR)-2-oxohexahydrothienol[3,4-d]-imidazole-4-valeric acid, although the trivial name biotin has become accepted in the meantime.

Vitamins, provitamins, and vitamin precursors from the groups A, B, E, and H are particularly preferred. Preferred in particular are nicotinamide, biotin, pantolactone, and/or panthenol.

The weight proportion of the vitamin(s), vitamin derivative(s), and/or the vitamin precursor(s) in terms of the total weight of the hair treatment agents is preferably about 0.001 to about 2% by weight, particularly preferably about 0.005 to about 1% by weight, and in particular about 0.01 to about 0.5% by weight.

Extracts that can be prepared from all parts of a plant are to be understood as suitable plant extracts. These extracts are typically produced by extraction of the entire plant. It can also be preferred in individual cases, however, to produce the extracts solely from the flowers and/or leaves of the plant. Suitable above all are the extracts from green tea, oak bark, stinging nettle, witch hazel, hops, chamomile, burdock, horsetail, whitethorn, lime blossom, lychee, almond, aloe vera, spruce needles, horse chestnut, sandalwood, juniper, coconut, mango, apricot, lemon, wheat, kiwi, melon, orange, grapefruit, sage, rosemary, birch, mallow, cuckoo flower, wild thyme, yarrow, thyme, melissa, restharrow, coltsfoot, marshmallow, ginseng, ginger root, Echinacea purpurea, Olea europaea, Boerhavia diffusa roots, Foeniculum vulgaris, and Apium graveolens.

The extracts from green tea, stinging nettle, witch hazel, chamomile, aloe vera, ginseng, Echinacea purpurea, Olea europea, and/or Boerhavia diffusa roots are particularly preferred for use in the compositions contemplated herein.

Water, alcohols, and mixtures thereof can be used as extracting agents to produce the cited plant extracts. Of the alcohols, low alcohols such as ethanol and isopropanol, but in particular polyhydric alcohols such as ethylene glycol and propylene glycol, are preferred, both as the sole extracting agent and in a mixture with water. Plant extracts based on water/propylene glycol in the ratio of 1:10 to 10:1 have proven to be particularly suitable.

The plant extracts can be used both in pure and diluted form. If they are used in diluted form, they conventionally contain about about 2 to about 80% by weight of active substance and as the solvent, the extracting agent or mixture of extracting agents used to obtain them.

The plant extracts can be used in the hair treatment agents contemplated herein (based on the total weight of the agents) preferably in an amount of about 0.01 to about 10% by weight, more preferably of about 0.05 to about 7.5% by weight, and in particular of about 0.1 to about 5% by weight.

Glycerol can be added to the hair cleaning and care agents separately in an amount of up to about 10% by weight (based on the total weight of the agent). However, it can also be a component of the aforesaid aqueous-alcoholic carrier.

It was found that the hair treatment agents contemplated herein are also suitable for use as an anti-dandruff formulation.

The total weight of anti-dandruff agents in terms of the total weight of the hair treatment agents can preferably be about 0.01 to about 10% by weight, more preferably about 0.025 to about 7.5% by weight, particularly preferably about 0.05 to about 5% by weight, and in particular about 0.075 to about 3% by weight.

Suitable anti-dandruff active agents can be selected from piroctone olamine, climbazole, zinc pyrithione, ketoconazole, salicylic acid, sulfur, selenium sulfide, tar preparations, undecenoic acid derivatives, burdock root extracts, poplar extracts, stinging nettle extracts, walnut shell extracts, birch extracts, willow bark extracts, rosemary extracts, and/or arnica extracts.

Climbazole, zinc pyrithione, and piroctone olamine are preferred.

Other active substances, auxiliary substances, and additives that can be contained in the hair treatment agents contemplated herein are, for example:
humectants,
perfume,
UV filters,
thickeners, such as gelatin or plant gum, for example, agar-agar, guar gum, alginates, xanthan gum, gum arabic, karaya gum, locust bean gum, flaxseed gums, dextrans, cellulose derivatives, e.g., methyl cellulose, hydroxyalkyl cellulose, and carboxymethyl cellulose, starch fractions and derivatives such as amylose, amylopectin, and dextrins, clays and phyllosilicates such as, e.g., bentonite, or fully synthetic hydrocolloids such as, e.g., polyvinyl alcohol, Ca, Mg, or Zn soaps,
structurants such as maleic acid and lactic acid,
dimethyl isosorbide,
cyclodextrins,
fiber-structure-improving active substances, particularly mono-, di-, and oligosaccharides such as, for example, glucose, galactose, fructose, fruit sugar, and lactose,
dyes for coloring the agent,
active substances such as bisabolol and/or allantoin,
complexing agents such as EDTA, NTA, β-alaninediacetic acid, and phosphonic acids,
ceramides. Ceramides are understood to be N-acylsphingosine (fatty acid amides of sphingosine) or synthetic analogs of such lipids (so-called pseudo-ceramides),
propellants such as propane-butane mixtures, N₂O, dimethyl ether, CO₂, and air,
antioxidants,
additional viscosity regulators such as salts (NaCl).

The agents contemplated herein are preferably so-called rinse-off products, i.e., are rinsed out of the hair again after a specific contact time. This contact time is preferably less than an hour; i.e., the user preferably does not leave the products in the hair until the next hair washing.

A further subject of the present invention is therefore a method for hair treatment in which an agent contemplated herein is applied to dry or damp hair, is left there for a time period of 30 to 300 seconds, and is rinsed out thereafter.

The agents contemplated herein lead to a considerably increased stability of artificial colors to the washing out of the color. Thus, chemically dyed hair can be washed much more often with the agents contemplated herein, without resulting in the unwanted bleeding or fading of the color.

A further subject of the present invention, therefore, is the use of the agents contemplated herein for reducing the washing out of color from chemically dyed hair.

The statements made about the agents contemplated herein apply mutatis mutandis in regard to preferred embodiments of the method contemplated herein and the use contemplated herein.

### Examples

All quantities are given in % by weight

### Hair Treatments

| | 1 | 2 | 3 | 4 | 5 | 6 |
|---|---|---|---|---|---|---|
| Cetearyl alcohol | 7.0 | 7.0 | 7.0 | 7.0 | 7.0 | 7.0 |
| Quaternium-87 | 0.9 | 0.9 | 0.9 | 0.9 | 0.9 | 0.9 |
| Cetrimonium chloride | 3.5 | 3.5 | 3.5 | 3.5 | 3.5 | 3.5 |
| Distearoylethyl Hydroxyethyl- | 2.0 | 2.0 | 2.0 | 2.0 | 2.0 | 2.0 |
| monium Methosulfate | | | | | | |
| Polyquaternium-10 | 0.1 | 0.1 | 0.1 | 0.1 | 0.1 | 0.1 |
| Baobab seed oil | 0.1 | 0.1 | 0.1 | 0.1 | 0.1 | 0.1 |
| Polyquaternium-67 | 0.2 | 0.2 | 0.2 | 0.2 | 0.2 | 0.2 |
| Hydrolyzed keratin | 0.5 | 0.5 | 0.5 | 0.5 | 0.5 | 0.5 |
| Glycol Distearate | 1.0 | 1.0 | 1.0 | 1.0 | 1.0 | 1.0 |
| Citric acid | 1.5 | 1.5 | 1.5 | 1.5 | 1.5 | 1.5 |
| Dimethicone | 1.0 | 1.0 | 1.0 | 1.0 | 1.0 | 1.0 |
| Calcium chloride | 1.0 | 1.0 | 1.0 | 1.0 | 1.0 | 1.0 |
| Amodimethicone | 0.5 | 0.5 | 0.5 | 0.5 | 0.5 | 0.5 |
| Propionic acid | 0.2 | 0.2 | 0.2 | 0.2 | 0.2 | 0.2 |
| o-Phenylphenol | — | 0.2 | 0.2 | 0.2 | 0.2 | 0.2 |
| o-Cymen-5-ol | — | 0.06 | 0.06 | 0.06 | 0.06 | 0.06 |
| Ethyl Lauroyl Arginate | — | 0.3 | 0.3 | 0.3 | 0.3 | 0.3 |
| Dimethyloxazolidine | — | 0.05 | 0.06 | 0.07 | 0.08 | 0.1 |
| Ethyl Lauroyl Arginate | — | 0.4 | 0.3 | 0.2 | 0.1 | 0.05 |
| Glutaraldehyde | — | 0.05 | 0.06 | 0.07 | 0.08 | 0.1 |
| Hexetidine | — | 0.05 | 0.06 | 0.07 | 0.08 | 0.1 |
| Phenoxyethanol | — | 0.05 | 0.05 | 0.05 | 0.05 | 0.05 |
| Phenoxyisopropanol | — | 0.05 | 0.1 | 0.25 | 0.05 | 0.05 |
| Undecylenic acid | — | 0.1 | 0.2 | 0.1 | 0.2 | 0.1 |
| Dye | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | To 100 |

### Hair Rinses

| | 1 | 2 | 3 | 4 | 5 | 6 |
|---|---|---|---|---|---|---|
| Cetearyl alcohol | 5.0 | 5.0 | 5.0 | 5.0 | 5.0 | 5.0 |
| Quaternium-87 | 0.75 | 0.75 | 0.75 | 0.75 | 0.75 | 0.75 |
| Behentrimonium Chloride | 1.0 | 1.0 | 1.0 | 1.0 | 1.0 | 1.0 |
| Behenoyl PG Trimonium | 1.5 | 1.5 | 1.5 | 1.5 | 1.5 | 1.5 |
| Chloride | | | | | | |
| Glycol Distearate | 1.0 | 1.0 | 1.0 | 1.0 | 1.0 | 1.0 |
| Distearoylethyl Hydroxy- | 0.3 | 0.3 | 0.3 | 0.3 | 0.3 | 0.3 |
| ethylmonium Methosulfate | | | | | | |
| Amodimethicone/ | 0.1 | 0.1 | 0.1 | 0.1 | 0.1 | 0.1 |
| Morpholinomethyl | | | | | | |
| Silsesquioxane Copolymer | | | | | | |
| Shea Butter | 1.5 | 1.5 | 1.5 | 1.5 | 1.5 | 1.5 |
| Lactic acid | 0.1 | 0.1 | 0.1 | 0.1 | 0.1 | 0.1 |
| Magnesium sulfate | 0.5 | 0.5 | 0.5 | 0.5 | 0.5 | 0.5 |
| Sodium propionate | 0.5 | 0.5 | 0.5 | 0.5 | 0.5 | 0.5 |
| Phenoxyethanol | — | 0.5 | 0.5 | 0.5 | 0.5 | 0.5 |
| Dimethyloxazolidine | — | 0.05 | 0.06 | 0.07 | 0.08 | 0.1 |
| Glutaraldehyde | — | 0.05 | 0.06 | 0.07 | 0.08 | 0.1 |
| Hexetidine | — | 0.05 | 0.06 | 0.07 | 0.08 | 0.1 |
| Ethyl Lauroyl Arginate | — | 0.05 | 0.05 | 0.05 | 0.05 | 0.05 |
| Sodium-o-phenylphenolate | — | 1.0 | 0.5 | 0.25 | 0.2 | 0.1 |
| o-Cymen-5-ol | — | 0.2 | 0.1 | 0.2 | 0.1 | 0.1 |
| Phenoxyisopropanol | — | 0.05 | 0.1 | 0.25 | 0.05 | 0.05 |
| Undecylenic acid | — | 0.1 | 0.2 | 0.1 | 0.2 | 0.1 |
| Dye | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | To 100 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment. It being understood that various changes may be made in the function and arrangement of elements described in an exemplary embodiment without departing from the scope as set forth in the appended claims.

1. A hair treatment agent, comprising:
a) at least one fatty alcohol,
b) at least one cationic surfactant,
c) at least one bivalent or trivalent metal salt,
d) at least one amino-functional silicone,
e) propionic acid and/or salts of propionic acid, and
f) at least one organic acid different from e).

2. The hair treatment agent according to claim 1 comprising a) fatty alcohol(s) of formula (II)
H3C—(CH2)k-CH2-OH (II)
in which k denotes integers from 4 to 28.

3. The hair treatment agent according to claim 1, comprising as b) behenyltrimethylammonium chloride.

4. The hair treatment agent according to claim 1, comprising as c) at least one divalent or trivalent metal salt chosen from the group of organic or inorganic copper, zinc, iron(II), calcium, magnesium, iron(III) and/or aluminum salts.

5. The hair treatment agent according to claim 1, comprising, based on its weight, about 0.01 to about 20% by weight of d) amino-functional silicone(s).

6. The hair treatment agent according to claim 1, comprising as d) amino-functional silicone(s) of the formula (Si-V) 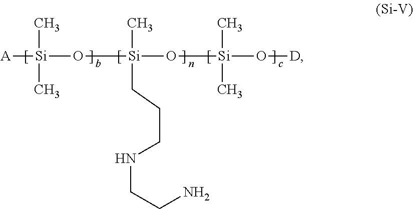
in which
A stands for a group —OH, —O—Si(CH₃)₃, —O—Si(CH₃)2OH, —O—Si(CH₃)₂OCH₃,
D stands for a group —H, —Si(CH₃)₃, —Si(CH₃)₂OH, —Si(CH₃)₂OCH₃,
b, n and c stand for integers between 0 and 1000,
provided that
i. n>0 and b+c>0; and
ii. at least one of the conditions: A=—OH or D=—H is met.

7. The hair treatment agent according to claim 1, comprising, based on its weight, about 0.001 to about 4% by weight of propionic acid.

8. The hair treatment agent according to claim 1, comprising as e) salt(s) chosen from the group of sodium propionate, potassium propionate, or calcium propionate.

9. The hair treatment agent according to claim 1, comprising as f) organic acid(s) chosen from the group of formic acid, oxalic acid, maleic acid, lactic acid, tartaric acid, or citric acid.

10. A method for treating hair, comprising:
applying a hair treatment agent to dry or moist hair, wherein the hair treatment agent comprises:
a) at least one fatty alcohol,
b) at least one cationic surfactant,
c) at least one bivalent or trivalent metal salt,
d) at least one amino-functional silicone,
e) propionic acid and/or salts of propionic acid, and
f) at least one organic acid different from e),
where the hair treatment is agent is left on the hair for a period of 30 to 300 seconds and then rinsed off.

11. The method of claim 10, wherein the hair treatment agent is applied to chemically dyed hair.

12. The hair treatment agent according to claim 2 comprising, based on the weight thereof, about 0.1 to about 20 wt. % of a) fatty alcohol(s) of formula (II).

13. The hair treatment agent according to claim 2, in which k denotes integers from 8 to 22.

14. The hair treatment agent according to claim 3, comprising, based on its weight, about 0.05 to about 20% by weight of behenyltrimethylammonium chloride as b).

15. The hair treatment agent according to claim 4, comprising, based on the weight thereof, about 0.01 to about 10 wt. % of c) divalent or trivalent metal salts.

16. The hair treatment agent according to claim 6, comprising, based on its weight, about 0.01 to about 20% by weight of d) the amino-functional silicone(s) of the formula (Si-V).

17. The hair treatment agent according to claim 8, comprising, based on its weight, about 0.001 to about 4% by weight of e) salt(s) chosen from the group of sodium propionate, potassium propionate, or calcium propionate.

18. The hair treatment agent according to claim 9, comprising based on its weight, about 0.001 to about 10% by weight of f) organic acid(s) chosen from the group of formic acid, oxalic acid, maleic acid, lactic acid, tartaric acid, or citric acid.

19. The hair treatment agent according to claim 1, comprising, based on the weight thereof, about 0.1 to about 20 wt. % of a) fatty alcohol(s), about 0.05 to about 20% by weight of behenyltrimethylammonium chloride as b), about 0.01 to about 10 wt. % of c) esterquat(s), about 0.01 to about 3 wt. % of d) cationic polysaccharide polymer(s), about 0.001 to about 5 wt. % of f) compounds of formula (I), and about 0.01 to about 10 wt. % of c) divalent or trivalent metal salts, about 0.01 to about 20% by weight of d) amino-functional silicone(s), about 0.001 to about 4% by weight of e) propionic acid or salts of propionic acid salt(s), and about 0.001 to about 10% by weight of f) organic acid(s).
